(19) United States
(12) Patent Application Publication
(10) Pub. No.: US 2022/0365536 A1
(43) Pub. Date: Nov. 17, 2022

(54) REAL-TIME SURFACE SCANNING AND ESTIMATION OF GROUND CHARACTERISTICS FOR GROUND COMPACTING WORK MACHINES

(71) Applicant: Deere & Company, Moline, IL (US)

(72) Inventors: Zimin W. Vilar, Dubuque, IA (US); Jonathan Spendlove, Bettendorf, IA (US); Francois Stander, Dubuque, IA (US); Giovanni A. Wuisan, Epworth, IA (US); Bradley C. Dauderman, Dubuque, IA (US)

(21) Appl. No.: 17/533,310

(22) Filed: Nov. 23, 2021

Related U.S. Application Data
(60) Provisional application No. 63/187,947, filed on May 13, 2021.

(51) Int. Cl. G05D 1/02; G01S 13/89; G01N 33/24; E02F 9/26; G06Q 10/06

(52) U.S. Cl. CPC G05D 1/0212; G01S 13/89; G01N 33/24; E02F 9/261; G06Q 10/0633

(57) ABSTRACT

System and methods are provided for dynamic characterization of an area to be worked using a work implement of a work machine. First real-time data (e.g., surface scan data) are collected in a forward direction via a first sensor external to or onboard the work machine, and second real-time data (e.g., surface scan data) are collected for at least a traversed portion of the work area via a second onboard sensor. Characteristic values of a ground material in the work area are determined based on at least the first and second data corresponding to a given surface, and outputs are generated corresponding to at least a determined amount of material needed to achieve target values for the work area, based on at least one of the characteristic values. Certain characteristic values based on the real-time data may be used to estimate, among other things, how many truck loads are still required for the work area.

### FIELD OF THE DISCLOSURE

The present disclosure relates generally to work machines such as for example self-propelled work machines which include implements mounted thereon for working the terrain. More particularly, the present disclosure relates to systems and methods for providing surface scanning capabilities with such work machines for dynamically generating real-time estimations regarding the surface characteristics of at least portions of a worksite.

### BACKGROUND

Many construction projects require extensive earthwork to ensure proper load bearing capacity, elevation, contouring and other characteristics for a given project. The earthwork for a given site of the project may involve importing and/or redistributing fill material. Fill material is generally delivered to the project site using transport vehicles such as for example articulated dump trucks (ADT's). Imported fill material may be redistributed about the site using self-propelled work machines such as bulldozers, graders, tractors, or the like. The redistributed fill material may then be compacted using more or more compactor machines, such as rollers or the like.

Prior to beginning earthwork, estimations are generally performed to determine the amount of imported fill material that will be needed for a given project. Surveying may be performed to determine the amount of fill material needed, generally measured in cubic yards. Complications arise when an estimator factors in bank cubic yardage, loose cubic yardage, the swell factor of a given fill material, and compaction factors of the given fill material to determine the expected compacted cubic yardage of the given fill material. A bank cubic yard, or BCY, is the calculation or measurement of one cubic yard of earth or rock (e.g., the fill material) in its natural state before it is removed from the ground and transported to the site of the project. A loose cubic yard, or LCY, is the measurement of the fill material after excavation. For example, when fill material is excavated from the ground, it is no longer compacted and further swells with the addition of air and/or water. This means that one bank cubic yard of fill material removed from the ground will almost always amount to more than one loose cubic yard of the same fill material. The swell factor is the percentage of increase in volume of the fill material (e.g., BCY) when it is excavated (e.g., the volume change from BCY to LCY). Swell of the fill material may also be referred to as fluff.

Once the loose fill material is transported to the site of the project, it is redistributed about the site, for example using bulldozers or the like. The compaction factor may include a light compaction factor (e.g., before use of rollers or equivalent compaction machines) and a heavy compaction factor (e.g., after use of the rollers or equivalent compaction machines). The compaction factor, whether heavy or light, is the percentage of decrease in volume of the fill material (e.g., LCY) when it is compacted (e.g., the change in volume from LCY to compacted cubic yards (CCY)). The compaction factor, like the swell factor, generally depends on the type of fill material.

Estimators may extrapolate approximate compactor factors based on the type of fill material, but these estimates are generally inaccurate. Alternatively, a before-scan or survey may be performed, the volume of fill material (e.g., LCY) moved to the site may be estimated, redistribution of the fill material may then be performed, and then an after-scan or survey may be performed to estimate the light compaction factor (e.g., before use of compaction machines) more accurately. Additionally, the redistributed fill material may be heavy compacted using one or more compaction machines prior to the after-scan or survey being performed in order to estimate the heavy compaction factor more accurately.

The estimator may further utilize the estimated light and heavy compaction factors to estimate a total number of truckloads of fill material necessary to complete the earthwork for a given site. Along these lines, estimators may also estimate the total number of bank cubic yards that will need to be excavated in order to provide an adequate amount of fill material for a given project.

Neither the estimated compaction factors nor the post-facto calculated light and heavy compaction factors provide real-time data or a reasonable equivalent for dynamically updating estimations, such that, for example, the remaining number of truckloads of fill material necessary to complete the earthwork for the given site or the remaining number of bank cubic yards that need to be excavated and transported to complete the earthwork for the given site.

### BRIEF SUMMARY

The current disclosure provides an enhancement to conventional systems, at least in part by introducing a novel system and method including real-time scanning of the worksite surface using equipment (e.g., a stereo camera, radar, LiDAR, and other optical sensors) mounted on a work machine such as a dozer or roller.

Exemplary systems and methods according to present disclosure may generate outputs corresponding to any estimations for characteristics such as bank yardage, swell factor, compaction factor, and the like, but substantially in real-time as opposed to the conventional tools. The real-time aspect of the surface scan and associated data processing further enables the estimation of compaction relative to banked yard density, or a direct and immediate comparison of a light compaction characteristic to a heavy compaction characteristic upon the ground being compacted by a work machine, and to utilize such estimations for further useful steps.

For example, systems and methods according to the present disclosure may utilize estimations as described above to predict an amount of material/number of transport vehicle cycles or loads required, such as in association with a given excavator, to meet any of various benchmarks.

In one embodiment, a method is disclosed herein for method for dynamic characterization of an area to be worked using at least one work implement of a work machine. First data may be collected for at least a forward portion of a work area relative to the work machine via at least a first sensor that may be external to or positioned onboard the work machine. Second data may be further collected for at least a traversed portion of the work area via at least a second sensor onboard the work machine. One or more characteristic values of a ground material in the work area may be determined based on first data for a specified portion of surface area and corresponding second data for the same specified surface area. Outputs may be generated corresponding to at least a determined amount of material needed to achieve a target value for the work area, based on at least one of the one or more characteristic values.

In one exemplary aspect according to the above-referenced embodiment, the first data may be collected via surface scans by the at least first sensor onboard the work machine, and the second data comprises position data collected via a global position sensor as the second sensor onboard the work machine, wherein the position data corresponds to a current elevation of a portion of the work machine corresponding to a traversed portion of the work area, and the one or more characteristic values of the ground material in the work area are further determined based on the current elevation of the portion of the work machine relative to the elevation of the at least a forward portion of the work area.

In another exemplary aspect according to the above-referenced embodiment, the first data may be collected via surface scans by the at least first sensor onboard the work machine, and the second data may be collected via surface scans by the at least second sensor onboard the work machine.

In another exemplary aspect according to the above-referenced embodiment, position data may further be collected via at least a third sensor onboard the work machine. A current elevation of the work machine is determined relative to an elevation of the at least a forward portion of the work area, wherein the one or more characteristic values of the ground material in the work area are further determined based on the current elevation of the work machine relative to the elevation of the at least a forward portion of the work area.

In another exemplary aspect according to the above-referenced embodiment, it is further provided to estimate a volume of material needed in the at least a forward portion of the work area and the at least a traversed portion of the work area to achieve the target value for the work area, based on at least one of the one or more characteristic values.

In another exemplary aspect according to the above-referenced embodiment, the first data for at least a forward portion of a work area may include: surface scan data collected prior to a discharge of loose fill material in the at least a forward portion of the work area; and surface scan data collected after the discharge of loose fill material in the at least a forward portion of the work area.

In another exemplary aspect according to the above-referenced embodiment, a compacted volume of the loose fill material may be estimated, and at least one of the one or more characteristic values further updated based on the second surface scan data upon traversal by the work machine of the area comprising the loose fill material.

In another exemplary aspect according to the above-referenced embodiment, a volume of material estimated as being needed in the at least a forward portion of the work area and the at least a traversed portion of the work area to achieve the target value for the work area (e.g., predetermined surface profile) may be based on the updated at least one of the one or more characteristic values.

In another exemplary aspect according to the above-referenced embodiment, a volume may be estimated of material added to the work area per transport vehicle load, and a number of transport vehicle loads required to achieve the target value for the work area may be predicted accordingly. The volume of material added to the work area per transport vehicle load may be estimated based at least in part on input signals from a payload weighing or measuring unit of the respective transport vehicle. As another example, the volume of material added to the work area per transport vehicle load may be estimated based at least in part on an estimated material carryback for the respective transport vehicle, wherein the material carryback is estimated using at least scanned images of a loading container of the transport vehicle.

In another exemplary aspect according to the above-referenced embodiment, a map may be accessed comprising three-dimensional data corresponding to at least a portion of the area to be worked. One or more desired discharge locations may be predicted in the at least a portion of the area to be worked, based at least in part on the estimated volume of material added to the work area per transport vehicle load and the predicted number of transport vehicle loads required to achieve the target value for the work area. Output signals may further be generated corresponding to the predicted one or more desired discharge locations to at least one transport vehicle.

In another exemplary aspect according to the above-referenced embodiment, for each of the at least one transport vehicle, a route may be generated for the transport vehicle between a detected current location thereof and at least one of the predicted one or more desired discharge locations, wherein the generated output signals to a respective transport vehicle correspond to route generated therefor.

In another exemplary aspect according to the above-referenced embodiment, the route for a respective transport vehicle may be generated based at least in part on received user input comprising at least one priority indicator with respect to the predicted one or more desired discharge locations, and/or on a detected payload weight.

In another exemplary aspect according to the above-referenced embodiment, the first sensor and the second sensor comprise one or more of: an image data source; an optical data source; and a radar sensor.

In another embodiment as disclosed herein, a system for dynamic characterization of an area to be worked using at least one work implement of a work machine may include: at least a first sensor onboard the work machine and configured to collect first surface scan data for at least a forward portion of a work area; at least a second sensor onboard the work machine and configured to collect second surface scan data for at least a traversed portion of the work area; and a controller functionally linked to the at least a first sensor and the at least a second sensor. The controller may be configured to direct the performance of operations according to the above-referenced method embodiment and optionally any one or more of the exemplary aspects thereof.

The controller of the above-referenced embodiment may typically be associated with the work machine itself. In other embodiments, the controller may be remote with respect to the work machine and take the form of a mobile computing device.

Numerous objects, features and advantages of the embodiments set forth herein will be readily apparent to those skilled in the art upon reading of the following disclosure when taken in conjunction with the accompanying drawings.

### BRIEF DESCRIPTION OF THE DRAWINGS

FIG. 1 is a perspective view of a tracked work machine incorporating an embodiment of a self-propelled work machine and method as disclosed herein.

FIG. 2 is a block diagram representing an exemplary control system for the work machine according to an embodiment of a system as disclosed herein.

FIG. 3 is a graphical diagram representing a worksite having compacted and loose portions of material, and a target profile requiring additional fill.

FIG. 4 is a flowchart representing an exemplary embodiment of a method as disclosed herein.

FIG. 5 is a flowchart representing an exemplary embodiment of a method as disclosed herein.

FIG. 6 is a flowchart representing supplemental optional features according to an exemplary embodiment of a method as disclosed herein.

### DETAILED DESCRIPTION

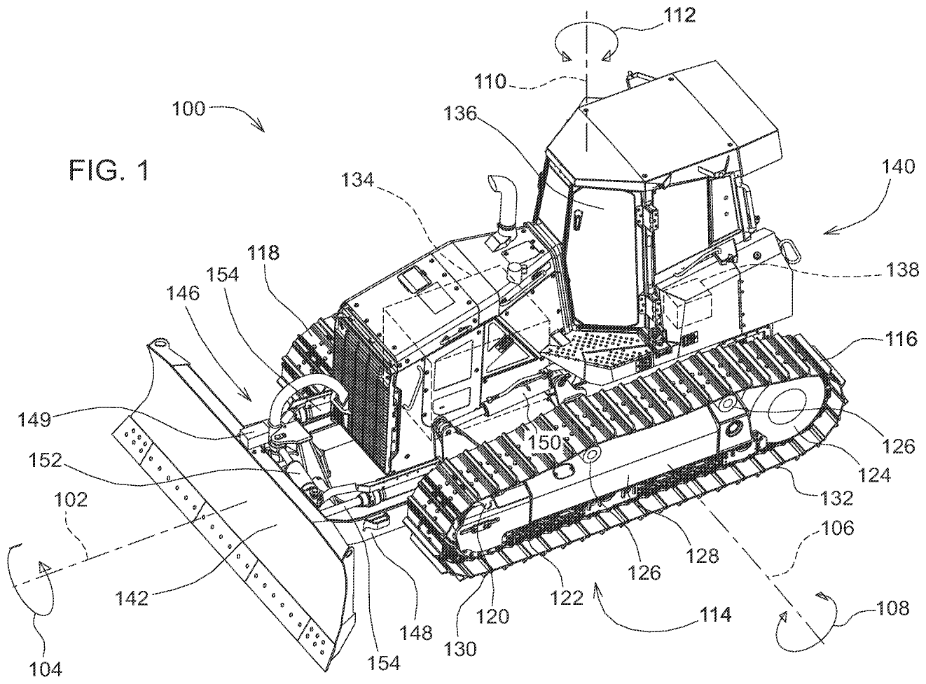

FIG. 1 is a perspective view of a work machine 100. In the illustrated embodiment, the work machine 100 is a crawler dozer, but may be any work machine with a ground-engaging blade or other work implement 142 such as a compact track loader, motor grader, scraper, skid steer, backhoe, and tractor, to name but a few examples. The work machine may be operated to engage the ground and grade, cut, and/or move material to achieve simple or complex features on the ground. While operating, the work machine may experience movement in three directions and rotation in three directions. A direction for the work machine may also be referred to with regard to a longitudinal direction 102, a latitudinal or lateral direction 106, and a vertical direction 110. Rotation for work machine 100 may be referred to as roll 104 or the roll direction, pitch 108 or the pitch direction, and yaw 112 or the yaw direction or heading.

An operator cab 136 may be located on the chassis 140. The operator cab 136 and the work implement 142 may both be mounted on the chassis 140 so that at least in certain embodiments the operator cab 136 faces in the working direction of the work implement 142, such as for example where the implement is front-mounted. A control station including a user interface 214 (not shown in FIG. 1) may be located in the operator cab 136. As used herein, directions with regard to work machine 100 may be referred to from the perspective of an operator seated within the operator cab 136: the left of work machine is to the left of such an operator, the right of work machine is to the right of such an operator, the front or fore of work machine 100 is the direction such an operator faces, the rear or aft of work machine is behind such an operator, the top of work machine is above such an operator, and the bottom of work machine is below such an operator.

The term “user interface” 214 as used herein may broadly take the form of a display unit 218 and/or other outputs from the system such as indicator lights, audible alerts, and the like. The user interface may further or alternatively include various controls or user inputs (e.g., a steering wheel, joysticks, levers, buttons) for operating the work machine 100, including operation of the engine, hydraulic cylinders, and the like. Such an onboard user interface may be coupled to a vehicle control system via for example a CAN bus arrangement or other equivalent forms of electrical and/or electro-mechanical signal transmission. Another form of user interface (not shown) may take the form of a display unit (not shown) that is generated on a remote (i.e., not onboard) computing device, which may display outputs such as status indications and/or otherwise enable user interaction such as the providing of inputs to the system. In the context of a remote user interface, data transmission between for example the vehicle control system and the user interface may take the form of a wireless communications system and associated components as are conventionally known in the art.

The illustrated work machine 100 further includes a control system including a controller 212 (further described below with respect to FIG. 3). The controller 212 may be part of the machine control system of the working machine, or it may be a separate control module. Accordingly, the controller 212 may generate control signals for controlling the operation of various actuators throughout the work machine 100, which may for example be hydraulic motors, hydraulic piston-cylinder units, electric actuators, or the like. Electronic control signals from the controller may for example be received by electro-hydraulic control valves associated with respective actuators, wherein the electro-hydraulic control valves control the flow of hydraulic fluid to and from the respective hydraulic actuators to control the actuation thereof in response to the control signal from the controller.

The controller 212 may include or be functionally linked to the user interface 214 and optionally be mounted in the operators cab 136 at a control panel.

The controller 212 is configured to receive input signals from some or all of various sensors 144, 149 associated with the work machine 100, which may include for example a set of one or more sensors 144 affixed to the chassis 140 and/or a set of one or more sensors 144 affixed to the work implement 142 of the work machine 100 and configured to provide signals indicative of an inclination (slope) of the chassis or the blade. In alternative embodiments, such sensors may not be affixed directly to the chassis but may instead be connected to the chassis 140 through intermediate components or structures, such as rubberized mounts. Such sensors 144 may be configured to provide at least a signal indicative of the inclination of the chassis 140 relative to the direction of gravity, or to provide a signal or signals indicative of other positions or velocities of the chassis, including its angular position, velocity, or acceleration in a direction such as the direction of roll 104, pitch 108, yaw 112, or its linear acceleration in a longitudinal direction 102, latitudinal direction 106, and/or vertical 110 direction. Sensors 144 may be configured to directly measure inclination, or for example to measure angular velocity and integrate to arrive at inclination, and may typically, e.g., be comprised of an inertial measurement unit (IMU) mounted on the chassis 140 and configured to provide at least a chassis inclination (slope) signal, or signals corresponding to the scope of the chassis 140, as inputs to the controller 212. Such an IMU may for example be in the form of a three-axis gyroscopic unit configured to detect changes in orientation of the sensor, and thus of the chassis 140 to which it is fixed, relative to an initial orientation.

In other embodiments, the sensors may include a plurality of GPS position sensors 232 fixed relative to the chassis 140 and/or the blade positioning unit, which can detect the absolute position and orientation of the work machine 100 within an external reference system, and can detect changes in such position and orientation.

The work machine 100 is supported on the ground by an undercarriage 114. The undercarriage 114 includes ground engaging units 116, 118, which in the present example are formed by a left track 116 and a right track 118 but may in certain embodiments be formed by alternative arrangements including wheeled ground engaging units, and provide tractive force for the work machine 100. Each track may be comprised of shoes with grousers that sink into the ground to increase traction, and interconnecting components that allow the tracks to rotate about front idlers 120, track rollers 122, rear sprockets 124 and top idlers 126. Such interconnecting components may include links, pins, bushings, and guides, to name a few components. Front idlers 120, track rollers 122, and rear sprockets 124, on both the left and right sides of the work machine 100, provide support for the work machine 100 on the ground. Front idlers 120, track rollers 122, rear sprockets 124, and top idlers 126 are all pivotally connected to the remainder of the work machine 100 and rotationally coupled to their respective tracks so as to rotate with those tracks. The track frame 128 provides structural support or strength to these components and the remainder of the undercarriage 114. In alternative embodiments, the ground engaging units 116, 118 may comprise, e.g., wheels on the left and right sides of the work machine.

Front idlers 120 are positioned at the longitudinal front of the left track 116 and the right track 118 and provide a rotating surface for the tracks to rotate about and a support point to transfer force between the work machine 100 and the ground. The left and right tracks 116, 118 rotate about the front idlers 120 as they transition between their vertically lower and vertically upper portions parallel to the ground, so approximately half of the outer diameter of each of the front idlers 120 is engaged with the respective left 116 or right track 118. This engagement may be through a sprocket and pin arrangement, where pins included in the left 116 and right tracks 118 are engaged by recesses in the front idler 120 so as to transfer force. This engagement also results in the vertical height of the left and right tracks 116, 118 being only slightly larger than the outer diameter of each of the front idlers 120 at the longitudinal front of the tracks. Forward engaging points 130 of the tracks 116, 118 can be approximated as the point on each track vertically below the center of the front idlers 120, which is the forward point of the tracks which engages the ground.

Track rollers 122 are longitudinally positioned between the front idler 120 and the rear sprocket 124 along the bottom left and bottom right sides of the work machine 100. Each of the track rollers 122 may be rotationally coupled to the left track 116 or the right track 118 through engagement between an upper surface of the tracks and a lower surface of the track rollers 122. This configuration may allow the track rollers 122 to provide support to the work machine 100, and in particular may allow for the transfer of forces in the vertical direction between the work machine and the ground. This configuration also resists the upward deflection of the left and right tracks 116, 118 as they traverse an upward ground feature whose longitudinal length is less than the distance between the front idler 120 and the rear sprocket 124.

Rear sprockets 124 may be positioned at the longitudinal rear of each of the left track 116 and the right track 118 and, similar to the front idlers 120, provide a rotating surface for the tracks to rotate about and a support point to transfer force between the work machine 100 and the ground. The left and right tracks 116, 118 rotate about the rear sprockets as they transition between their vertically lower and vertically upper portions parallel to the ground, so approximately half of the outer diameter of each of the rear sprockets 124 is engaged with the respective left or right track 116, 118. This engagement may be through a sprocket and pin arrangement, where pins included in the left and right tracks are engaged by recesses in the rear sprockets 124 to transfer force. This engagement also results in the vertical heights of the tracks being only slightly larger than the outer diameter of each of the rear sprockets 124 at the longitudinal back or rear of the respective track. The rearmost engaging point 132 of the tracks can be approximated as the point on each track vertically below the center of the rear sprockets, which is the rearmost point of the track which engages the ground. In this embodiment, each of the rear sprockets 124 may be powered by a rotationally coupled hydraulic motor so as to drive the left track 116 and the right track 118 and thereby control propulsion and traction for the work machine 100. Each of the left and right hydraulic motors may receive pressurized hydraulic fluid from a hydrostatic pump whose direction of flow and displacement controls the direction of rotation and speed of rotation for the left and right hydraulic motors. Each hydrostatic pump may be driven by an engine 134 (or equivalent power source) of the work machine and may be controlled by an operator in the operator cab 136 issuing commands which may be received by the controller 212 and communicated to the left and right hydrostatic pumps. In alternative embodiments, each of the rear sprockets may be driven by a rotationally coupled electric motor or a mechanical system transmitting power from the engine.

Top idlers 126 are longitudinally positioned between the front idlers 120 and the rear sprockets 124 along the left and right sides of the work machine 100 above the track rollers 122. Similar to the track rollers, each of the top idlers may be rotationally coupled to the left track 116 or the right track 118 through engagement between a lower surface of the tracks and an upper surface of the top idlers. This configuration may allow the top idlers to support the tracks for the longitudinal span between the front idler and the rear sprocket and prevent downward deflection of the upper portion of the tracks parallel to the ground between the front idler and the rear sprocket.

The undercarriage 114 is affixed to, and provides support and tractive effort for, the chassis 140 of the work machine 100. The chassis is the frame which provides structural support and rigidity to the work machine, allowing for the transfer of force between the work implement 142 (e.g., blade) and the left track 116 and right track 118. In this embodiment, the chassis is a weldment comprised of multiple formed and joined steel members, but in alternative embodiments it may be comprised of any number of different materials or configurations.

The blade of the present example is a work implement 142 which may engage the ground or material, for example to move material from one location to another and to create features on the ground, including flat areas, grades, hills, roads, or more complexly shaped features. In this embodiment, the blade of the work machine 100 may be referred to as a six-way blade, six-way adjustable blade, or power-angle-tilt (PAT) blade. The blade may be hydraulically actuated to move vertically up or down (“lift”), roll left or right (“tilt”), and yaw left or right (“angle”). Alternative embodiments may utilize a blade with fewer hydraulically controlled degrees of freedom, such as a 4-way blade that may not be angled or actuated in the direction of yaw 112.

The work implement 142 is movably connected to the chassis 140 of the work machine 100 through a linkage 146 which supports and actuates the blade and is configured to allow the blade to be lifted (i.e., raised or lowered in the vertical direction 110) relative to the chassis. The linkage 146 includes a c-frame 148, a structural member with a C-shape positioned rearward of the work implement 142, with the C-shape open toward the rear of the work machine 100. The work implement 142 may be lifted (i.e., raised or lowered) relative to the work machine 100 by the actuation of lift cylinders 150, which may raise and lower the c-frame 148. The work implement 142 may be tilted relative to the work machine 100 by the actuation of a tilt cylinder 152, which may also be referred to as moving the blade in the direction of roll 104. The work implement 142 may be angled relative to the work machine 100 by the actuation of angle cylinders 154, which may also be referred to as moving the work implement 142 in the direction of yaw 112. Each of the lift cylinders 150, tilt cylinder 152, and angle cylinders 154 may be a double acting hydraulic cylinder.

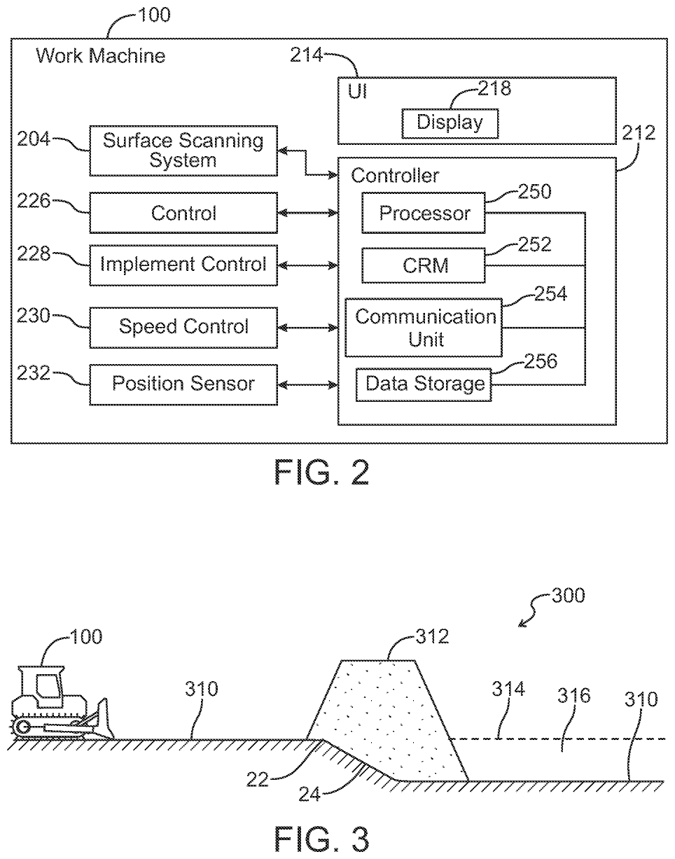

As schematically illustrated in FIG. 2, the work machine 100 in an embodiment as disclosed herein includes a control system including a controller 212. The controller 212 may be part of the machine control system of the work machine 100, or it may be a separate control module.

As referenced above, the controller 212 is configured to receive input signals from a surface scanning system 204 which may in one example include stereo cameras and collectively defining an imaging system. In the alternative or in addition, an imaging system may include one or more of an infrared camera, a video camera, a stereoscopic camera, a PMD camera, or the like. One of skill in the art may appreciate that a surface scanning system 204 within the scope of the present disclosure may include high resolution light detection and ranging (LiDAR) scanners, radar detectors, laser scanners, and the like. The number and orientation of said scanners in a surface scanning system 204 may vary in accordance with the type of work machine 100 and relevant applications, but may at least be provided with respect to areas forward and/or rearward of the work machine 100 and accordingly configured to capture data associated with relevant surroundings proximate the work machine 100.

While the figures generally illustrate data collection in the context of onboard sensors configured to perform surface scans, it may be understood that unless specifically stated otherwise additional sensors external to the work machine 100 may be functionally linked to the controller 212 and within the scope of a surface scanning system 204. For example, in certain embodiments surface scans could be performed by a crawler preceding a compactor as the work machine 100 (using a visual scanning device or a GPS assisted position sensor) and then relayed to the compactor as the work machine 100 (thereby generating its forward scan), or the data may be collected using an external scanning device mounted on for example an unmanned aerial vehicle (UAV, or drone).

The position and size of an image region recorded by a respective camera as a data source in surface scanning system 204 may depend on the arrangement and orientation of the camera and the camera lens system, in particular the focal length of the lens of the camera. One of skill in the art may further appreciate that image data processing functions may be performed discretely at a given image data source if properly configured, but also or otherwise may generally include at least some image data processing by the controller or other downstream data processor. For example, image data from any one or more surface scanning data sources may be provided for three-dimensional point cloud generation, image segmentation, object delineation and classification, and the like, using image data processing tools as are known in the art in combination with the objectives disclosed.

The controller 212 of the work machine 100 may be configured to produce outputs, as further described below, to a user interface 214 associated with a display unit 218 for display to the human operator. The controller 212 may be configured to receive inputs from the user interface 214, such as user input provided via the user interface 214. Not specifically represented in FIG. 2, the controller 212 of the work machine 100 may in some embodiments further receive inputs from and generate outputs to remote devices associated with a user via a respective user interface, for example a display unit with touchscreen interface. Data transmission between for example the vehicle control system and a remote user interface may take the form of a wireless communications system and associated components as are conventionally known in the art. In certain embodiments, a remote user interface and vehicle control systems for respective work machines 100 may be further coordinated or otherwise interact with a remote server or other computing device for the performance of operations in a system as disclosed herein.

The controller 212 may in various embodiments, as part of the control system of FIG. 2 and further in line with the above-referenced disclosure, be configured to generate control signals for controlling the operation of respective actuators, or signals for indirect control via intermediate control units, associated with a machine steering control system 226, a machine work implement control system 228, and an engine speed control system 230. The control systems 226, 228, 230 may be independent or otherwise integrated together or as part of a machine control unit in various manners as known in the art. The controller 212 may for example generate control signals for controlling the operation of various actuators, such as hydraulic motors or hydraulic piston-cylinder units (not shown), and electronic control signals from the controller 212 may actually be received by electro-hydraulic control valves associated with the actuators such that the electro-hydraulic control valves will control the flow of hydraulic fluid to and from the respective hydraulic actuators to control the actuation thereof in response to the control signal from the controller 212.

A position sensor 232 as conventionally known in the art such as for example a global positioning system (GPS) transceiver or the like may further be provided and communicatively linked to the controller 212.

The controller 212 includes or may be associated with a processor 250, a computer readable medium 252, a communication unit 254, and data storage 256 such as for example a database network. It is understood that the controller 212 described herein may be a single controller having some or all of the described functionality, or it may include multiple controllers wherein some or all of the described functionality is distributed among the multiple controllers.

Various operations, steps or algorithms as described in connection with the controller 212 can be embodied directly in hardware, in a computer program product such as a software module executed by the processor 250, or in a combination of the two. The computer program product can reside in RAM memory, flash memory, ROM memory, EPROM memory, EEPROM memory, registers, hard disk, a removable disk, or any other form of computer-readable medium 252 known in the art. An exemplary computer-readable medium 252 can be coupled to the processor 250 such that the processor 250 can read information from, and write information to, the memory/storage medium 252. In the alternative, the medium 252 can be integral to the processor 250. The processor 250 and the medium 252 can reside in an application specific integrated circuit (ASIC). The ASIC can reside in a user terminal. In the alternative, the processor 250 and the medium 252 can reside as discrete components in a user terminal.

The term “processor” 250 as used herein may refer to at least general-purpose or specific-purpose processing devices and/or logic as may be understood by one of skill in the art, including but not limited to a microprocessor, a microcontroller, a state machine, and the like. A processor 250 can also be implemented as a combination of computing devices, e.g., a combination of a DSP and a microprocessor, a plurality of microprocessors, one or more microprocessors in conjunction with a DSP core, or any other such configuration.

The communication unit 254 may support or provide communications between the controller 212 and external communications units, systems, or devices, and/or support or provide communication interface with respect to internal components of the work machine 100. The communications unit may include wireless communication system components (e.g., via cellular modem, WiFi, Bluetooth or the like) and/or may include one or more wired communications terminals such as universal serial bus ports.

The data storage 256 as further described below may, unless otherwise stated, generally encompass hardware such as volatile or non-volatile storage devices, drives, electronic memory, and optical or other storage media, as well as in certain embodiments one or more databases residing thereon.

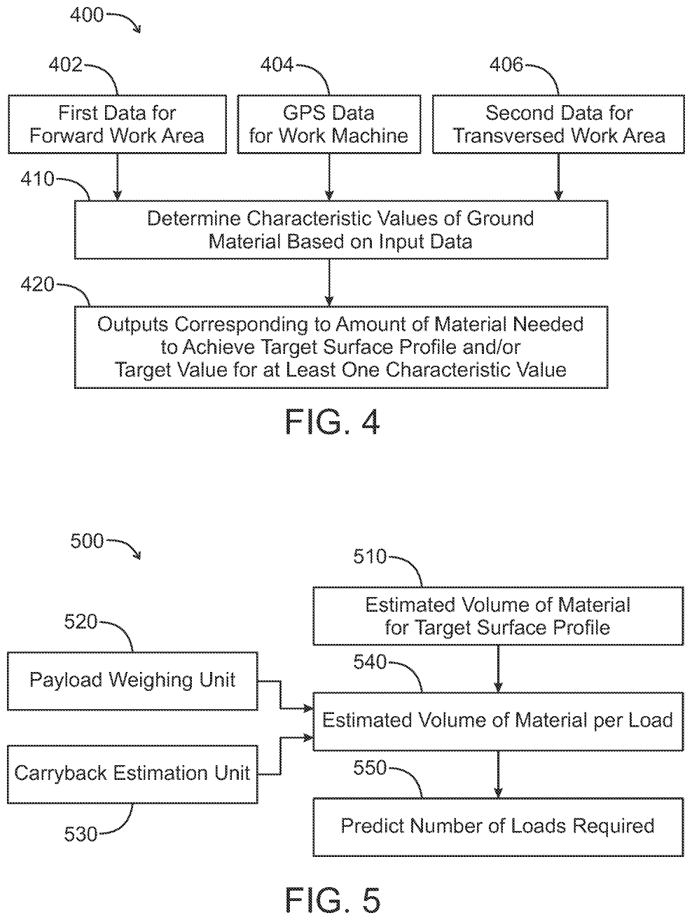

Referring next to FIG. 4, an exemplary method 400 of the present disclosure may next be described. The method 400 includes at least a step 410 of determining characteristic values of ground material in a work area based on various inputs, substantially in real-time.

One set of inputs may be provided as first data 402 of a surface in a forward work area or otherwise stated a forward portion of the work area. In various embodiments, the first data may be provided via surface scans of the forward work area. The term “forward work area” or equivalents as used herein may refer for example to at least a portion of the work area generally in front of the work machine 100 when the work machine is travelling in a forward direction. As previously noted, the scanned data may be provided via an image data source (e.g., stereo camera), optical sensor, radar sensor, etc. The first scanned data as collected in the forward portion of the work area may include for example a collection of surface scan data prior to a discharge of loose fill material in the forward work area and a collection of surface scan data after discharge of the loose fill material in the forward work area.

Another set of inputs may be provided as second data 406 of a surface in a traversed work area or otherwise stated a traversed portion of the work area. In various embodiments, the second data 506 may be provided via surface scans of the traversed work area. The term “traversed work area” or equivalents as used herein may typically refer for example to at least a portion of the work area generally rearward of the work machine when the work machine is travelling in a forward direction and having been traversed by the work machine. The sensor(s) providing the second scanned data may be different from the sensor(s) providing the first scanned data, or they may include one or more overlapping sensors.

Another (or an alternative) set of inputs may be provided as position data 404 from a position sensor such as for example a GPS transceiver mounted or otherwise provided on the work machine 100. The position data 404 may for example be utilized with corresponding first surface scan data 402 to determine a current elevation of the work machine 100 relative to a surface forward of the work machine, and/or may be utilized with corresponding second surface scan data 406 to determine a current elevation of the work machine 100 relative to a surface previously traversed by the work machine.

Relative elevation (Δz) values may be utilized among other inputs to determine the characteristic values of the ground material, as an indication for example of the swell factor, compaction ratio, or the like for a particular surface upon which for example the work machine has traversed.

In at least one particular example wherein the work machine 100 is a drum roller compactor or an equivalent, a surface scanning sensor may be utilized to generate surface scan data 402 while a position sensor is utilized to generate position data 404, without requiring a separate surface scanning sensor or the equivalent for generating data 406 in the traversed work area. The forward scan along with high precision GPS signals and known work machine body kinematics may be sufficient to determine a surface height of the rear drum (as being the last point wherein the work machine 100 contacts the ground surface and therefore equating to the elevation of the traversed work area) and therefore to determine the compaction ratio.

The various data points may in various embodiments be provided as time-series data or the like, wherein for example first scanned data 402 and corresponding second scanned data 406 for the same portion of the ground surface may be directly compared and processed by the system based on for example a ground speed of the work machine to match the respective data.

In another step 420 of the method 400, output signals may be provided corresponding to an amount of material needed to be added for achieving one or more targets in the work area. Referring to a simplified diagram of a work area as illustrated in FIG. 3, a work machine 100 may traverse a compacted ground surface of the work area 310, a portion of which is prior to the loose fill 312 and generally corresponds to a desired or target surface profile 314. A volume difference 316 between the current ground surface and the target surface profile 314 will need to be filled, for example by operations including the spreading and compacting of the loose fill 312. In the illustrated example, the loose fill 312 is discharged over a slope 24 between the portion of the compacted ground surface 310 corresponding to the target surface profile 314 and a portion of the current ground surface that still needs to be filled, and further overlaps with an upper lip 22 of the slope 24 of the surface 310. By scanning the relevant areas prior to traverse by the work machine (e.g., dozer, roller), further scanning the same areas after traverse, and making calculations substantially in real-time with respect to the second scans for a given area, relevant characteristics of the spread and (at least lightly) compacted surface may be determined and utilized for important further estimations. Otherwise stated, in various embodiments a system as disclosed herein may be capable of measuring any number of surface characteristics as are conventionally known (e.g., bank yardage, swell factor, compaction factor), as well as any number of derivative characteristics therefrom, but the calculations may be made substantially in real-time upon traversal of the ground when utilizing the multiple surface scan inputs as disclosed herein and further dynamically updating estimates based on for example current scan results as opposed to predicted scan results.

In an exemplary embodiment as disclosed herein and as represented in FIG. 5, another method 500 enabled by the method 400 and system configuration described above includes estimating (step 510) a volume of material required to achieve a target surface profile 314 for a work area, estimating (step 540) a volume of material that is transported to the work area on a per load basis, and accordingly predicting (step 550) a number of transport vehicle loads that will be required to achieve the target surface profile 314. The initial estimates may for example be provided in view of current surface scans (and/or input data from a topographic map of the worksite) and further in view of conventional mathematical models and assumptions, but the accuracy of these estimates may be improved over time with real-time feedback comprising comparisons of the ground surface before it was traversed by the work machine to the same ground surface afterwards.

In an embodiment, the estimated volume of material that is transported with each load may be estimated without any feedback from the transport vehicles. For example, after numerous iterations it may be mathematically determined how much an initial estimated volume of material required differs from a current estimated volume of material required, further in view of the number of loads transported.

The estimated volume of material that is transported with each load may however in other embodiments further account for inputs from the transport vehicle itself, such as for example via a payload weighing or measuring unit as is known in the art. Volume estimations directly based on inputs from a payload weighing or measuring unit (520) may lack a high degree of accuracy on their own, given for example the uncertainty regarding the state/density of the material as it is being transported, but in various embodiments may usefully be implemented among other inputs or as a confirmation of other inputs. As another example, inputs from the payload weighing or measuring unit after discharge of material may be provided for carryback estimation (530) as part of the overall volume estimation, in other words such that a remaining amount of material in the loading container after discharge is accounted for in the volume estimation. Material carryback may further or in the alternative be estimated based on scans of the loading container as compared to a known profile of the loading container when completely empty.

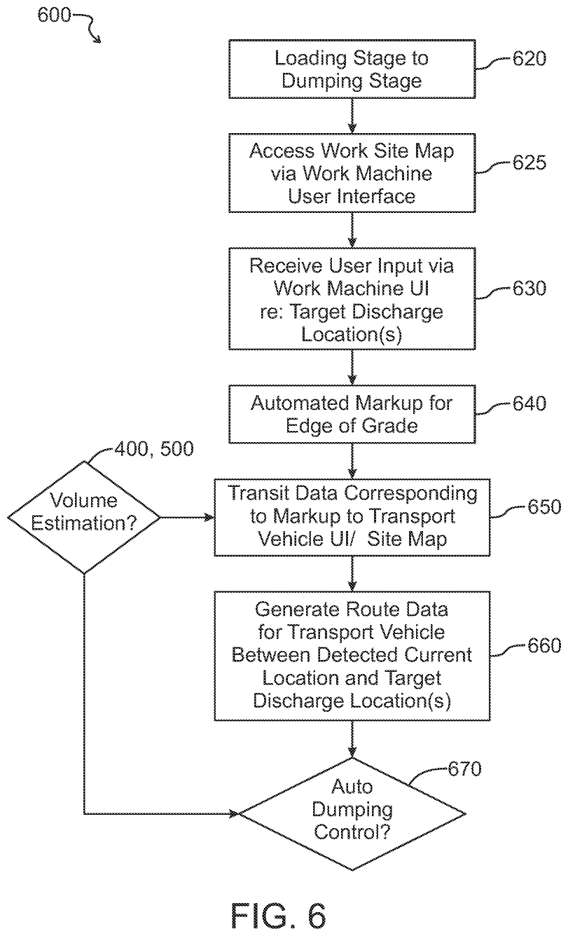

Referring next to FIG. 6, an embodiment as disclosed herein may further include steps in a method 600 to optionally enable the prediction and/or selection of desired material discharge locations within the work area.

Such an embodiment may be initiated upon detecting the transition from a loading stage to a discharging (dumping) stage for a particular transport vehicle (step 620). It may be determined that the transport vehicle is in motion from a loading area to a prospective discharge (dumping) area in a worksite, for example based on values obtained from a payload weighing unit 520 further in view of vehicle speed inputs.

In a second step 630, a work machine 100 such as for example a tracked dozer may include a first user interface 214 that is configured to selectively access a map comprising three-dimensional data corresponding to at least a portion of a worksite to be worked. In one embodiment, the predicted number of transport vehicle loads required to achieve a target value for the work area (as previously disclosed) may be implemented along with a predetermined work plan associated with the map to predict target discharge locations for the transport vehicles. A sample set of target discharge locations may be predicted automatically by the system and presented to a user for selective modification via an onboard display unit 218 which includes visual information corresponding to the worksite map, as well as providing interface tools such as touchscreen inputs in order for the operator to select one or more areas as target discharge locations 30 on the worksite map where it is preferred that material be discharged by the transport vehicle 10. In certain embodiments, the user interface 214 may enable further inputs such as for example varying levels of priority (e.g., closest, lowest) with respect to each of the one or more selected areas. In other embodiments, the automatic prediction of target discharge locations may be omitted or otherwise disabled, wherein selection of target discharge locations is fully manual in implementation.

The user interface 214 may be configured to display a first image layer associated with real-time inputs from an image data source 204 mounted on the work machine 100 and corresponding to surroundings of the work machine, and to further display a second image layer associated with inputs from the accessed map, wherein the second image layer comprises an overlay or superposed visual elements with respect to the first image layer and corresponding to three-dimensional information regarding the worksite. In an embodiment, the second image layer (or a third image layer) may further comprise visual elements corresponding to user inputs (e.g., breadcrumbs) that correspond to selected target discharge locations in the context of the displayed surroundings and three-dimensional information.

In an embodiment, the user interface 214 for receiving user input corresponding to the preferred discharge location does not need to be mounted onboard the work machine 100 but may be provided on a mobile computing device or the like, whether associated with an operator of the work machine 100 or another authorized user associated with the worksite.

Upon receiving the user input, the system and method 600 may in some embodiments continue (i.e., step 640) by modifying the selected discharge locations with respect to an edge of the grade. For example, an operator selection for one location relative to the edge of a grade may be automatically corrected by the system based on an estimated size of a load 20 of material being carried to the site by the transport vehicle, a type of material, a severity of the detected grade, or the like. Generally stated, it may be desirable and accordingly predetermined within the system that an edge of the material pile upon discharge overlaps with the lip 22 of the grade 24 by a specified minimal distance.

With sufficient information received from the user interface 214 or otherwise derived from the user input in association with a target discharge location, the method 600 may continue in step 650 by transmitting output signals comprising data corresponding to the target discharge location to a transport vehicle. For example, each of a plurality of transport vehicles may be assigned one or more of the predicted and/or selected target discharge locations, wherein corresponding output signals are generated to the respective transport vehicles.

In an embodiment, a smart mapping system as disclosed herein may comprise a common mapping interface associated with respective user interfaces on the work machine 100 and on certain transport vehicles, wherein selections made by an operator of the work machine 100 with respect to a target discharge location are processed and transmitted in substantially real-time for visual implementation on a user interface of the transport vehicle.

In other embodiments, certain of the transport vehicles may be provided with a mapping or other visual system associated with the worksite that is independent from the mapping system of the work machine 100, in which case user inputs from the work machine 100 may be translated by a system as disclosed herein from a coordinate system relevant to the work machine (i.e., associated with the perspective of the operator when providing the user inputs via the user interface) into a coordinate system independent of the work machine, and then further as needed translated by the system into another coordinate system relevant to the transport vehicle (i.e., associated with the perspective of a driver of the transport vehicle via an associated display unit or otherwise for the specification of route instructions).

In step 660, the driver of the transport vehicle may be provided with data corresponding to a route generated by the system, including position targeting outputs in the form of for example instructions displayed via the respective worksite mapping unit of the transport vehicle or via the common (“smart”) mapping system of the vehicles collectively. The route may preferably be determined to account for any intervening terrain and associated objects between the target discharge location and/or target stopping location and the current position of the transport vehicle.

As a prerequisite for generation of the map and route for the transport vehicle, the system may first need to detect or otherwise determine a current location of the transport vehicle, for example using GPS data and machine-to-machine communications. Alternatively, information corresponding to a distance between the transport vehicle and the work machine 100 may be provided using time-of-flight imaging techniques, a beacon transmitter or RFID tag, or image classification and associated processing of contours of the loading container.

In one example, the driver of the transport vehicle may simply follow visual indicators generated on the displayed map and follow a displayed route or, using for example a back-up camera system, line up the sight to a specified parking location relative to the target stopping location where a current load 20 should preferably be dumped/discharged. As previously noted, the targeting system may preferably line up the discharged load 20 of material so that the discharged pile overlaps (by a predetermined or otherwise minimal distance) the edge 22 of the grade 24. Such an embodiment may in an embodiment include coordination of the controller 212 associated with the work machine 100 and a controller associated with the transport vehicle, for example via bidirectional data communication using a communications network. For example, using an overhead (i.e., top-down) view or a front camera view as a first image layer displayed on an onboard user interface of the transport vehicle, a superposed second image layer may be generated to inform the driver of the transport vehicle of information corresponding to the determined route, the target discharge location, the target stopping location, and/or the like, wherein the driver simply drives at least the loading container into position.

In another example, an automated dumping control mode (step 660) may optionally be provided wherein the transport vehicle is at least partially controlled to direct movement of at least the loading container thereof to a specified target stopping location relative to the target discharge location. Such an embodiment may for example include coordination of the controller 212 associated with the work machine 100 (or other system control unit) and a controller associated with the transport vehicle, for example via bidirectional data communication using a communications network. As one example, when the transport vehicle has reached a specified or threshold distance from the target discharge location and/or target stopping location, the output signals may be generated from the controller 212 or other system control unit to the transport vehicle to inform the driver that an auto-park system is available for the remaining portion of the determined route. The operator may then acknowledge activation of the auto park mode, for example via an onboard user interface, wherein a steering control system for the transport vehicle automatically takes over steering using control data associated with or otherwise derived from the output signals from the work machine controller 212. In various embodiments, the driver may still be responsible for one or more driving aspects, including for example braking, even during the auto-park mode but a fully autonomous feature may also be contemplated within the scope of the present disclosure.

In an embodiment, sensors onboard the work machine 100 and/or other sensors or alternative inputs such as for example from the payload weighing unit 520 of the transport vehicle may be implemented to estimate a volume of material required to fill at least a portion of the worksite, and/or a volume of material currently or previously discharged in one or more locations relative to the at least a portion of the worksite. This may account for example on the relative elevation of low portions of the unfilled portion of the worksite with respect to completed portions, and further accounting in some embodiments for changes in the scanned terrain from before grading to after grading (i.e., upon light compaction).

The system and method 600 may further optionally include estimating a volume of carryback material 530 remaining in a loading container of the transport vehicle after a material discharge with respect to the one or more locations relative to the at least a portion of the worksite, and accounting for the estimated volume of carryback material in estimating the volume of material discharged in the one or more locations relative to the at least a portion of the worksite. For example, one or more image data sources mounted on the transport vehicle may be configured to scan a bottom of the loading container when the loaded material has theoretically been fully discharged, wherein a profile of the remaining material relative to an expected profile for the empty loading container may be processed to estimate the carryback material. As previously noted, image data sources as disclosed herein may include radar, lidar, and equivalent sensing units that can generate point clouds of data as opposed to just conventional images. The carryback estimation module may optionally further account for inputs from the payload weighing unit 520.

Volume estimations as noted above, for the amounts being discharged by the transport vehicles relative to an expected amount, or for an amount of material required to fill at least a relevant portion of the worksite, may optionally be provided as inputs (step 635) with respect to the driver assistance inputs on the user interface of the transport vehicle, for example to account for a size of the expected discharge if/when dynamically modifying the target discharge location or to potentially change a relative priority among a selected plurality of target discharge locations.

Such volume estimations may also, or in the alternative, be provided as inputs (step 635) with respect to the automated discharge control mode of step 660, for example to account for a size of the expected discharge if/when dynamically modifying the target discharge location, or to potentially change a relative priority among a selected plurality of target discharge locations.

As used herein, the phrase “one or more of,” when used with a list of items, means that different combinations of one or more of the items may be used and only one of each item in the list may be needed. For example, “one or more of” item A, item B, and item C may include, for example, without limitation, item A or item A and item B. This example also may include item A, item B, and item C, or item B and item C.

Thus, it is seen that the apparatus and methods of the present disclosure readily achieve the ends and advantages mentioned as well as those inherent therein. While certain preferred embodiments of the disclosure have been illustrated and described for present purposes, numerous changes in the arrangement and construction of parts and steps may be made by those skilled in the art, which changes are encompassed within the scope and spirit of the present disclosure as defined by the appended claims. Each disclosed feature or embodiment may be combined with any of the other disclosed features or embodiments.

What is claimed is:

1. A method for dynamic characterization of an area to be worked using at least one work implement of a work machine, the method comprising:
collecting first data for at least a forward portion of a work area relative to the work machine via at least a first sensor external to or onboard the work machine;
collecting second data for at least a traversed portion of the work area via at least a second sensor onboard the work machine;
determining one or more characteristic values of a ground material in the work area based on at least first data for a specified area and corresponding second data for the specified area; and
generating outputs corresponding to at least a determined amount of material needed to achieve a target value for the work area, based on at least one of the one or more characteristic values.

2. The method of claim 1, wherein:
the first data are collected via surface scans by the at least first sensor onboard the work machine,
the second data comprise position data collected via a global position sensor as the second sensor onboard the work machine, the position data corresponding to a current elevation of a portion of the work machine corresponding to a traversed portion of the work area, and
the one or more characteristic values of the ground material in the work area are further determined based on the current elevation of the portion of the work machine relative to the elevation of the at least a forward portion of the work area.

3. The method of claim 1, wherein the first data are collected via surface scans by the at least first sensor onboard the work machine, and wherein the second data are collected via surface scans by the at least second sensor onboard the work machine.

4. The method of claim 3, further comprising:
collecting position data via at least a third sensor onboard the work machine, and
determining a current elevation of the work machine relative to one or more of an elevation of the at least a forward portion of the work area and an elevation of the at least a traversed portion of the work area,
wherein the one or more characteristic values of the ground material in the work area are further determined based on the current elevation of the work machine relative to the one or more of an elevation of the at least a forward portion of the work area and an elevation of the at least a traversed portion of the work area.

5. The method of claim 1, further comprising:
estimating a volume of material needed in the at least a forward portion of the work area and the at least a traversed portion of the work area to achieve the target value for the work area, based on at least one of the one or more characteristic values.

6. The method of claim 5, wherein collecting first data for at least a forward portion of a work area comprises:
collecting surface scan data prior to a discharge of loose fill material in the at least a forward portion of the work area; and
collecting surface scan data after the discharge of loose fill material in the at least a forward portion of the work area.

7. The method of claim 6, comprising:
estimating a compacted volume of the loose fill material, and updating at least one of the one or more characteristic values based on the second data upon traversal by the work machine of the area comprising the loose fill material.

8. The method of claim 7, wherein the estimation of a volume of material needed in the at least a forward portion of the work area and the at least a traversed portion of the work area to achieve the target value for the work area is based on the updated at least one of the one or more characteristic values.

9. The method of claim 5, further comprising:
estimating a volume of material added to the work area per transport vehicle load; and
predicting a number of transport vehicle loads required to achieve the target value for the work area.

10. The method of claim 9, wherein the volume of material added to the work area per transport vehicle load is estimated based at least in part on input signals from a payload weighing or measuring unit of the respective transport vehicle.

11. The method of claim 10, wherein the volume of material added to the work area per transport vehicle load is estimated based at least in part on an estimated material carryback for the respective transport vehicle.

12. The method of claim 9, further comprising:
accessing a map comprising three-dimensional data corresponding to at least a portion of the area to be worked;
predicting one or more desired discharge locations in the at least a portion of the area to be worked, based at least in part on the estimated volume of material added to the work area per transport vehicle load and the predicted number of transport vehicle loads required to achieve the target value for the work area; and
generating output signals corresponding to the predicted one or more desired discharge locations to at least one transport vehicle.

13. The method of claim 12, further comprising, for each of the at least one transport vehicle, generating a route for the transport vehicle between a detected current location thereof and at least one of the predicted one or more desired discharge locations, wherein the generated output signals to a respective transport vehicle correspond to route generated therefor.

14. The method of claim 13, wherein the route for a respective transport vehicle is generated based at least in part on received user input comprising at least one priority indicator with respect to the predicted one or more desired discharge locations, and/or on a detected payload weight.

15. A system for dynamic characterization of an area to be worked using at least one work implement of a work machine, the system comprising:
at least a first sensor external to or onboard the work machine and configured to collect first data for at least a forward portion of a work area relative to the work machine;
at least a second sensor onboard the work machine and configured to collect second data for at least a traversed portion of the work area;
a controller functionally linked to the at least a first sensor and the at least a second sensor and configured to
determine one or more characteristic values of a ground material in the work area based on at least first data for a specified area and corresponding second data for the specified area; and
generate outputs corresponding to at least a determined amount of material needed to achieve a target value for the work area, based on at least one of the one or more characteristic values.

16. The system of claim 15, wherein:
the first data are collected via surface scans by the at least first sensor onboard the work machine,
the second data comprise position data collected via a global position sensor as the second sensor onboard the work machine, the position data corresponding to a current elevation of a portion of the work machine corresponding to a traversed portion of the work area, and
the one or more characteristic values of the ground material in the work area are further determined based on the current elevation of the portion of the work machine relative to the elevation of the at least a forward portion of the work area.

17. The system of claim 15, wherein the first data are collected via surface scans by the at least first sensor onboard the work machine, and wherein the second data are collected via surface scans by the at least second sensor onboard the work machine.

18. The system of claim 17, further comprising:
at least a third sensor onboard the work machine and configured to collect position data,
wherein the controller is configured to determine a current elevation of the work machine relative to one or more of an elevation of the at least a forward portion of the work area and an elevation of the at least a traversed portion of the work area,
wherein the one or more characteristic values of the ground material in the work area are further determined based on the current elevation of the work machine relative to the one or more of an elevation of the at least a forward portion of the work area and an elevation of the at least a traversed portion of the work area.

19. The system of claim 15, wherein the controller is further configured to estimate a volume of material needed in the at least a forward portion of the work area and the at least a traversed portion of the work area to achieve the target value for the work area, based on at least one of the one or more characteristic values.

20. The system of claim 19, wherein:
the first data for at least a forward portion of a work area comprises:
surface scan data collected prior to a discharge of loose fill material in the at least a forward portion of the work area; and
surface scan data collected after the discharge of loose fill material in the at least a forward portion of the work area, and. the controller is configured to:
estimate a compacted volume of the loose fill material, and
update at least one of the one or more characteristic values based on the second surface scan data upon traversal by the work machine of the area comprising the loose fill material.
